(19) United States
(12) Patent Application Publication
(10) Pub. No.: US 2026/0263762 A1
(43) Pub. Date: Sep. 10, 2026

(54) BALLOON CATHETERS AND ASSOCIATED METHODS

(71) Applicant: Accumed Radial Systems, LLC, Farmington Hills, MI (US)

(72) Inventor: Joseph R Korotko, Livonia, MI (US)

(21) Appl. No.: 19/168,900

(22) PCT Filed: Mar. 27, 2024
(86) PCT No.: PCT/US2024/021725

§ 371 Date: Sep. 25, 2025

Related U.S. Application Data
(60) Provisional application No. 63/454,778, filed on Mar. 27, 2023.
(60) Provisional application No. 63/511,634, filed on Jun. 30, 2023.

(51) Int. Cl. A61M 25/10

(52) U.S. Cl. CPC A61M 25/1029; A61M 25/1002; A61M 2025/105; A61M 2025/1079

(57) ABSTRACT

A method of manufacturing a balloon catheter includes providing a balloon having a helical portion including a radially outer portion and a radially inner portion at least partially bounding a lumen, and electrospinning a cover onto at least one of the radially outer portion and the radially inner portion

### CROSS-REFERENCED TO RELATED APPLICATION

This application claims priority to U.S. Provisional Application No. 63/454,778, filed Mar. 27, 2023 and U.S. Provisional Application No. 63/511,634, filed Jun. 30, 2023.

### BACKGROUND

Balloon catheters have various applications, such as for vascular treatment of the coronary arteries. In general, balloon catheters are insertable into the patient's vasculature and maneuverable to the desired location.

The term “catheter” may refer collectively to a wide range of medical devices that are inserted into the body to (1) diagnose a medical condition; (2) treat a medical condition; (3) deliver nourishment; or (4) deliver medicine. The term “catheter” may be used more specifically to refer to a tube inserted into the body of a patient for the purposes of (a) removing material from a location in the body of a patient and/or (b) delivering medicinal and/or nourishing material to a specific location within the body of a patient. Catheters can be used in a variety of locations for a variety of purposes within the body of a patient. Catheterization procedures are commonly involved in the diagnosis and treatment of the cardiovascular system, the excretory system, and other similar systems of a patient.

### SUMMARY

A method of manufacturing a balloon catheter, according to an example of this disclosure, includes providing a balloon having a helical portion including a radially outer portion and a radially inner portion at least partially bounding a lumen, and electrospinning a cover onto at least one of the radially outer portion and the radially inner portion.

In a further example, the method includes electrospinning the cover onto both the radially outer portion and the radially inner portion.

In a further example, the helical portion includes a plurality of coils having axial space therebetween, and the cover extends across the axial spaces.

In a further example, the method includes coating the cover with a therapeutic product.

In a further example, the helical portion includes a plurality of coils providing the radially inner portion and the radially outer portion and having axial space therebetween, and the cover extends across the axial spaces.

In a further example, the method includes coating the cover with a therapeutic product.

In a further example, the cover has a thickness of about 10-about 100 microns.

In a further example, the cover includes at least one of polyethylene, polypropylene, polyurethane, nylon, and polyester.

A system for creating a lumen, according to an example of this disclosure, includes a tubular balloon, the tubular balloon operable in a low-profile operating mode and a high-profile operating mode. The example system includes an electrospun cover constraining the tubular balloon in a generally helical shape. The tubular balloon has a first diameter in a low-profile operating mode and a second diameter in a high-profile operating mode, and the second diameter is larger than the first diameter.

In a further example, the tubular balloon provides a radially outer portion and a radially inner portion, the radially inner portion at least partially bounding the lumen, and the cover is electrospun onto at least one of the radially outer portion and the radially inner portion.

In a further example, the cover is electrospun onto both the radially outer portion and the radially inner portion.

In a further example, the tubular balloon includes a plurality of coils providing the radially inner portion and the radially outer portion and having axial space therebetween, and the cover extends across the axial spaces.

In a further example, the tubular balloon includes a plurality of coils providing the radially inner portion and the radially outer portion and having axial space therebetween, and the cover extends across the axial spaces.

In a further example, the cover includes at least one of polyethylene, polypropylene, polyurethane, nylon, and polyester.

In a further example, the cover is coated with a therapeutic product.

In a further example, the cover has a thickness of about 10-about 100 microns.

In a further example, the cover includes at least one of polyethylene, polypropylene, polyurethane, nylon, and polyester.

In a further example, the cover is coated with a therapeutic product.

In a further example, the cover has a thickness of about 10- about 100 microns.

In a further example, the cover includes at least one of polyethylene, polypropylene, polyurethane, nylon, and polyester.

These and other features may be best understood from the following specification and drawings, the following of which is a brief description.

### BRIEF DESCRIPTION OF THE DRAWINGS

FIG. 1 shows an example balloon catheter system.

FIGS. 2A-B show a detail view of a balloon catheter of the balloon catheter system of FIG. 1.

FIG. 2C shows a cross sectional view of a portion of an example tube.

FIG. 3 shows a detail view of a movement mechanism of the balloon catheter system of FIG. 1.

FIG. 4 shows another example balloon catheter system.

FIG. 5 shows the example balloon catheter system of FIG. 4 with a balloon catheter in a low-profile operating mode.

FIG. 6 shows the example balloon catheter system of FIG. 4 with a balloon catheter in a high-profile operating mode.

FIGS. 7A-7D schematically show an example balloon catheter.

FIGS. 8A-8D illustrate an example angioplasty method.

### DETAILED DESCRIPTION

In general, a balloon catheter may be flexible enough to be inserted into and maneuvered through a patients' vasculature which includes various curves and corners, and in particular may include consecutive opposing curves that are known as tortuous areas. The balloon catheter also may be flexible enough to be atraumatic so as not cause any damage during use. If the catheter is too stiff, it may be difficult to use in tortuous areas and it may dissect or perforate an artery while being maneuvered. However, at the same time, the balloon catheter may be stiff enough to be pushed or inserted into the patient. If a catheter is too flexible it may be difficult to navigate in tortuous areas.

The systems disclosed herein may create a lumen within a body of a patient to facilitate the use of a medical device, such as the use of a catheter in a blood vessel. The systems may facilitate catheterization by creating additional “working space” (e.g., the lumen) at a desired location within the body of a patient. The additional space may be created by transitioning from a low-profile operating mode into a high-profile operating mode. The additional space may enable the use of other medical devices by overcoming problems of conventional access such as vessel tortuosity or insignificant stenoses. The systems may enable a balloon angioplasty catheter or stent catheter to be inserted through the passageway or tunnel of the lumen past the access problems and onto a desired location. In some examples, the lumens created allow the arteries to not be occluded when the systems are inserted, allowing for increased insertion time relative to traditional balloon catheters.

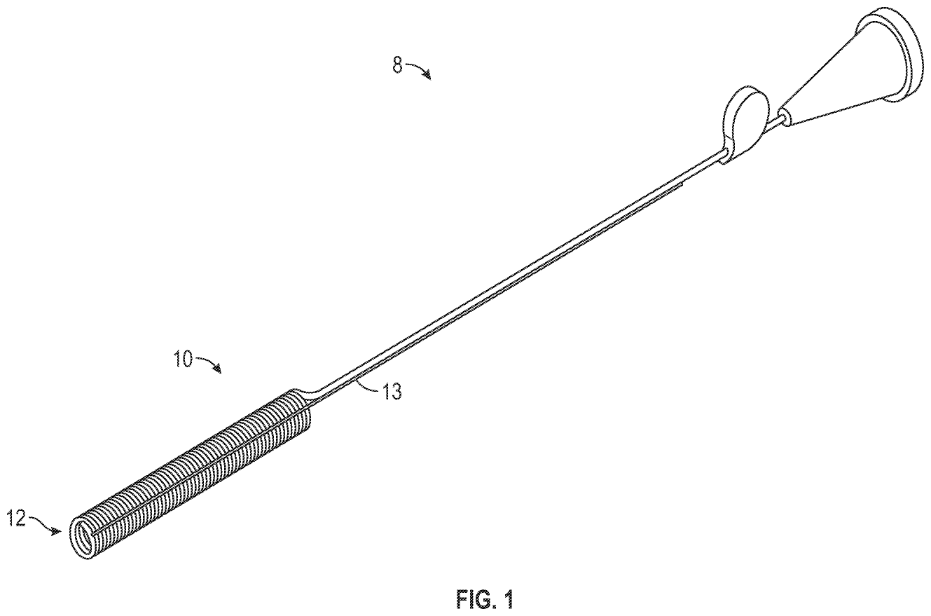

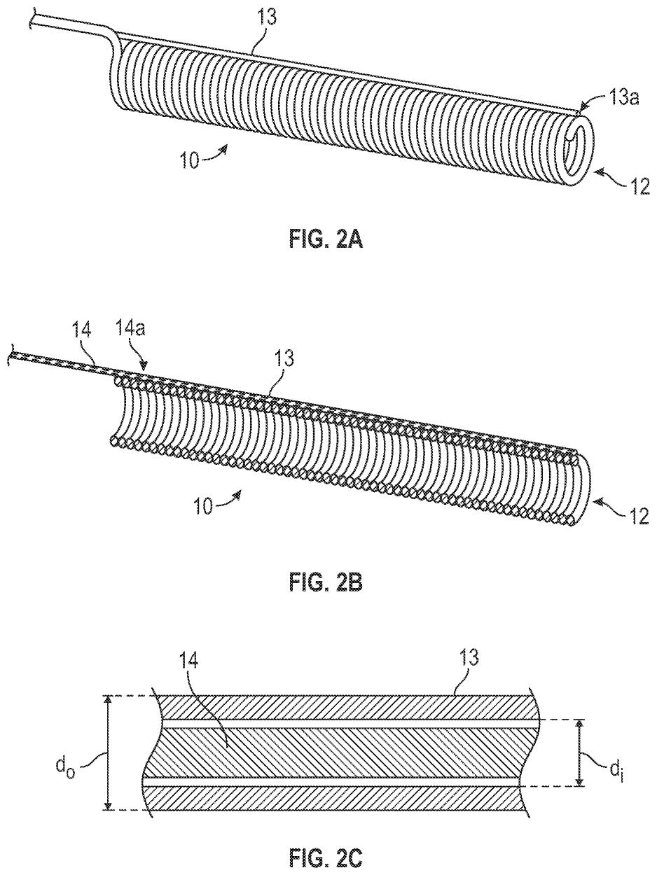

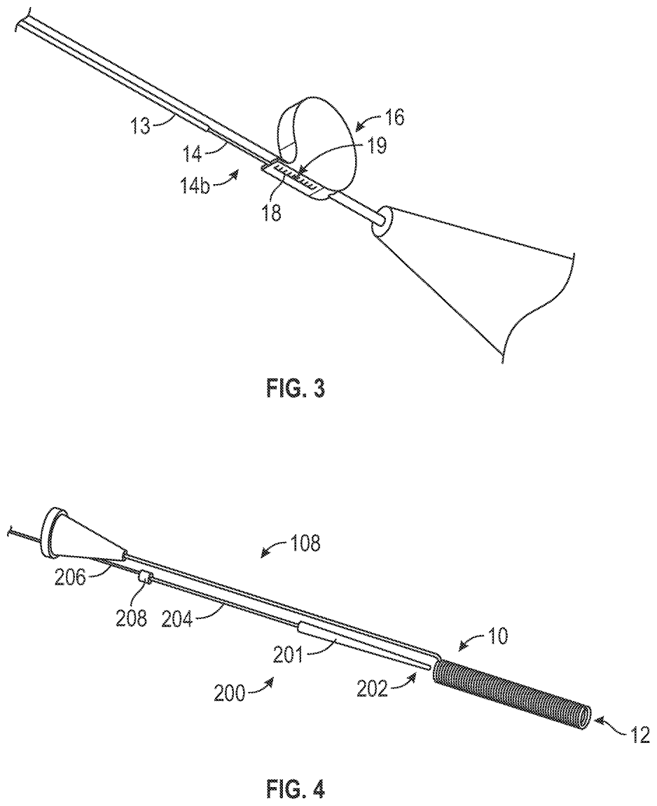

FIG. 1 schematically shows an example balloon catheter system 8 with varying flexibility. In some examples, the balloon catheter system 8 may be used in balloon angioplasty procedures. FIGS. 2A and 2B show a detail view of the example balloon catheter system 8. The balloon catheter system 8 includes a balloon catheter 10 with a lumen 12 therein. In some examples, the balloon catheter system 8 may also include other features such as a valve for interfacing with other medical devices, like an inflation system.

In some examples, as shown, a tube 13 is situated alongside the balloon catheter 10 and spans the length or substantially the length of the balloon catheter 10. In the example of FIGS. 1-2B, the tube 13 is situated outside of the lumen 12, on an outer surface of the balloon catheter 10. In other examples, however, the tube 13 may be situated in the lumen 12 along an inner surface of the balloon catheter 10.

The tube 13 may be connected to the balloon catheter 10 by any suitable means. In some examples, the tube 13 may be continuous with or integral with the balloon catheter 10. In some examples, the tube 13 has about the same flexibility as the balloon catheter 10. In particular examples, the tube 13 is of the same material as the balloon catheter 10. The tube 13 may be hollow and configured to receive a support wire 14 within the inner diameter of the tube 13 (seen in FIG. 2B). The tube 13 may protect and guide the support wire 14 and likewise assist in preventing the support wire 14 from poking or otherwise damaging the vasculature of the patient. In some examples, a distal end 13a of the tube 13 is closed off.

In one example the tube 13 is a polyimide tube. As shown in FIG. 2C, the polyimide tube 13 may have an inside diameter dᵢ of about 0.008-about 0.011 inch and an outside diameter dₒ of about 0.011-about 0.014 inch. In a particular example, the inside diameter dᵢ may be about 0.008-inch and the outside diameter dₒ is about 0.011-inch, and the support wire 14 is a 0.007-inch diameter support wire.

The support wire 14 may be a metal wire, such as a medical grade stainless steel (SST) wire. The support wire 14 may be made from 304 SST or 304L SST, in some examples. Other examples, such as cables or polymeric materials, are contemplated for the support wire 14. The support wire 14 may have a diameter of about 0.007-about 0.010 inch in some examples.

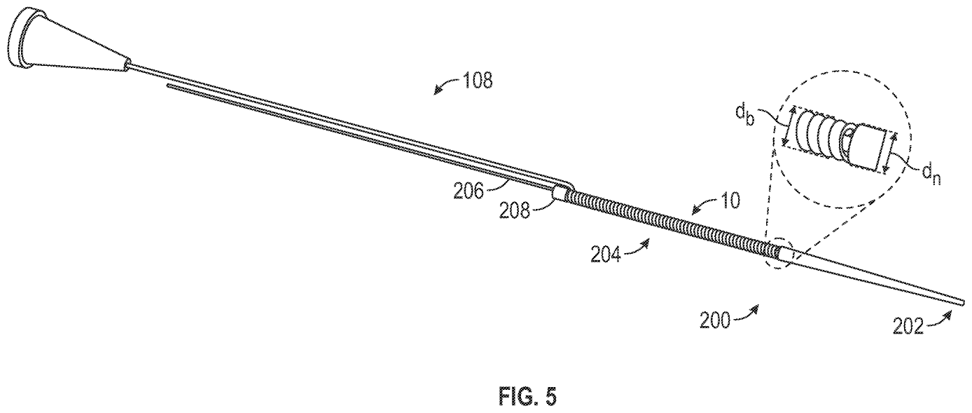

In some examples, the support wire 14 has variable flexibility. For instance, a distal end 14a (FIG. 2B) of the support wire 14 may have a different stiffness than the opposite, proximal end 14b (FIG. 3). In one example, the distal end 14a may be more flexible than the proximal end 14b. The support wire 14 can be tapered so that the flexibility/stiffness changes with the diameter of the taper, or may include multiple materials/material structures (such as braids, cables, or coils) that have different stiffnesses.

In some applications, a medical professional may need a stiffer catheter to be able to insert the catheter to the correct location. Or, alternately, a more flexible catheter may be needed. Being able to move a wire 14 in-and-out of the balloon enables the medical professional to change the stiffness of the distal end of the catheter. Further, if the wire 14 has variable stiffness, then when the wire 14 is completely inserted into the balloon the balloon can have variable stiffness, which may be an advantage to inserting the catheter to the correct location.

As will be discussed in more detail below, the support wire 14 may be selectively positioned within the tube 13. For example, the support wire 14 may at various times be positioned within the tube 13 to extend along the entirety of the balloon catheter 10 (i.e., the support wire 14 is fully extended), only portions of the balloon catheter 10 (i.e. the support wire 14 is partly extended), or none of the balloon catheter 10 (i.e. the support wire 14 is fully retracted). For instance, the support wire 14 may be positioned to extend along only the proximal half of the balloon catheter 10. The support wire 14 may increase the stiffness of the balloon catheter 10 selectively in only those areas alongside which it extends. The flexibility/stiffness of the balloon catheter 10 can therefore be selectively modulated in some examples.

The balloon catheter 10 may take any form. In the example of the foregoing figures, the balloon catheter 10 is a helical balloon that can be inflated to provide the lumen 12. However, other forms are contemplated.

In one example, the balloon catheter system 8 includes a retractor 16 for extending and retracting the support wire 14 relative to the balloon catheter 10, such as a lever or turn knob (best shown in FIG. 3). The retractor 16 may include markings that indicate the position of the support wire 14 relative to the balloon catheter 10. For instance, an icon 18 could be included on the retractor 16, such as on a handle, of the balloon catheter for positioning the support wire 14. An arrow 19 on the retractor 16 may point to a location on the icon which would indicate the position of the support wire 14 relative to the balloon portion of the balloon catheter. In particular, the markings of the icon 18 may correspond to a location of a distal end 14a of the support wire 14 relative to the balloon catheter 10.

The balloon catheter system 8 may include a guidewire lumen and/or a rapid exchange (Rx) feature (not shown). The guidewire lumen and/or Rx feature would be separate and distinct from the tube 13/support wire 14.

The balloon catheter system 8 may be used as follows. A medical provider may, partially or fully, advance the support wire 14 into the tube 13 to increase the stiffness of the balloon catheter 10. The balloon catheter 10 may then be inserted into the body and vasculature of the patient 14. During the insertion, the medical provider may modulate the stiffness of the balloon catheter 10 depending on the features of the vasculature, such as tortuous areas, by extending/retracting the support wire 14 as discussed above. In some examples, the medical provider may completely retract the support wire 14 from the balloon catheter system 8 and replace it with another support wire having a different desired stiffness, thereby providing a different desired stiffness to the balloon catheter 10.

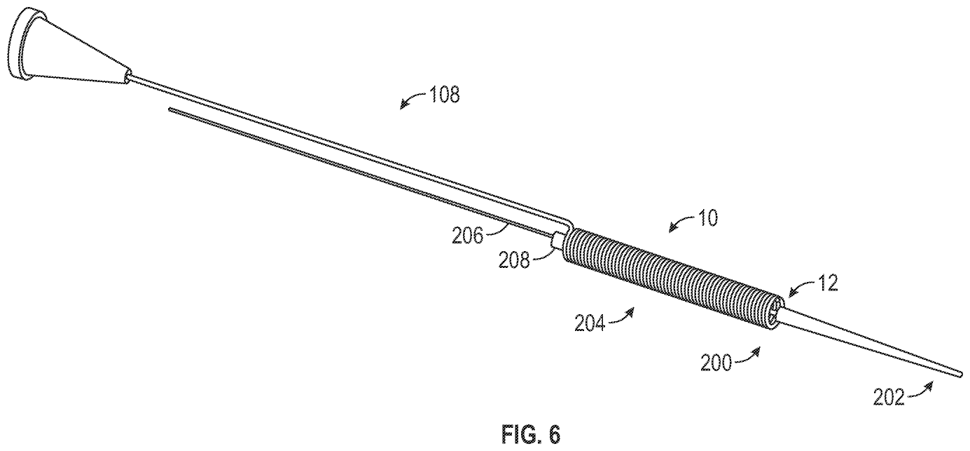

FIG. 4 shows another example balloon catheter system 108. The balloon catheter system 108 may include any of the features of the balloon catheter system 8 discussed above, and vice versa. In some examples, the balloon catheter system 108 may be used in balloon angioplasty procedures. The balloon catheter system 108 includes a balloon catheter 10 and a nosecone 200. The nosecone 200 includes a conical portion 201 having a pointed tip 202 spaced distally from an introducer 204. The conical portion 201 may be generally a soft or “rubberized” atraumatic (blunted) cone-shaped tapered tip that acts as the leading or distal point of the balloon catheter assembly 108 as shown in FIG. 4. The nosecone 200 thus facilitates inserting the balloon catheter assembly 108 into the patient to the target location. The introducer 204 may be a rod that extends proximally from the conical portion 201 and allows positioning of the nosecone 200 relative to the balloon catheter 10. In some examples, the nosecone 200 includes a pullwire 206 attached to, and extending proximally from, the introducer 204. The pullwire 206 is configured to remain accessible to the medical provider using the balloon catheter system 108 when it is inserted in the body of a patient, so that the nosecone 200 can be removed independently of the balloon catheter 10 as will be discussed in more detail below.

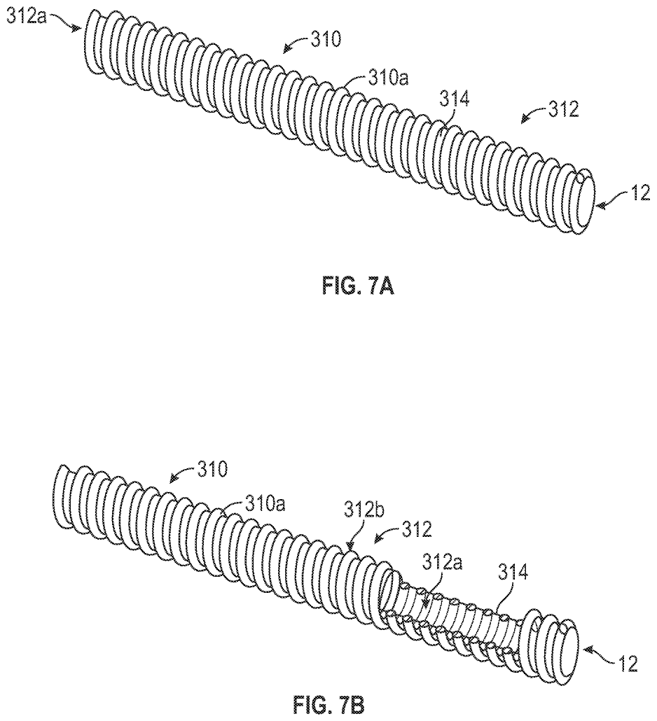

The nosecone 200 may be configured to be received in the lumen 12 of the balloon catheter 10. When the balloon catheter 10 is in a deflated or low-profile operating mode it may collapse around a portion of the nosecone 200. In a particular example shown in FIG. 5, the nosecone 200 may be arranged so that the collapsed balloon catheter 10 is arranged around the introducer 204.

The conical portion 201 of the nosecone 200 may have a maximum diameter dn that is about the same size or slightly larger than a collapsed or low-profile diameter db of the balloon catheter 10. In some examples, the diameter dn is about 0.040-about 0.042 inch. In some examples, the introducer 204 includes a collar 208 at its proximal end. The collar 208 has a diameter that is about the same size or slightly larger than the low-profile diameter db of the balloon catheter 10, and may have the same diameter as the maximum diameter dn of the conical portion 201. The balloon catheter 10 is thus trapped between the conical portion 201 and the collar 208 when it is in the low-profile operating mode in this example.

The nosecone 200 may be configured to interface with other medical devices used with balloon catheters. For example, the balloon catheter system 108 may be configured to interface with a coronary guidewire, including a 0.014-inch diameter coronary guidewire in some examples. In this example, the nosecone 200 includes a 0.016 inch diameter lumen therethrough configured to receive the guidewire.

The balloon catheter assembly 108 may be used as follows. The nosecone 200 may be situated in the balloon catheter 10 as shown in FIG. 5. The balloon catheter assembly 108 may then be inserted into a patient's vasculature to a desired location with the balloon catheter 10 in the low-profile operating mode. As discussed above, the nosecone 200 may include features to keep the balloon catheter 10 in place on the nosecone 200 during the insertion, such that the balloon catheter 10 and the nosecone 200 move together.

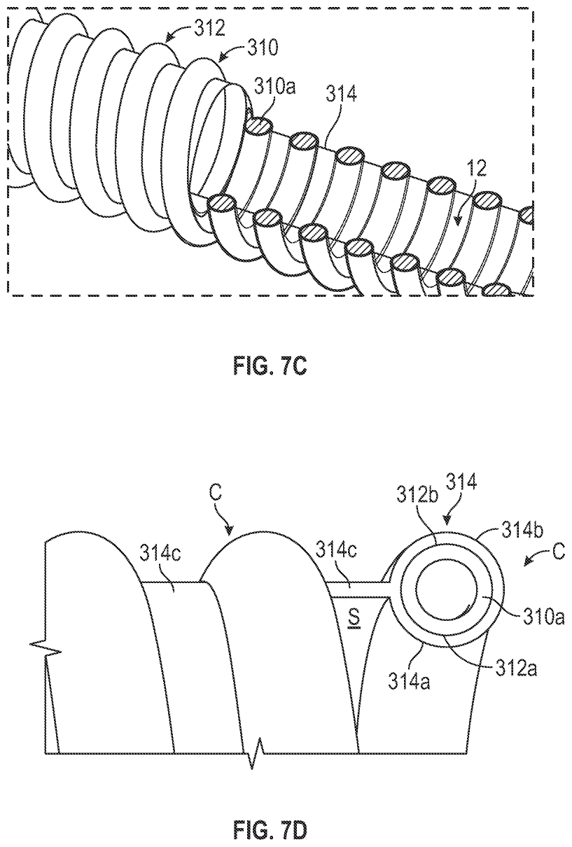

Once the balloon catheter 10 is in the desired location, it is transitioned to a high-profile operating mode such as by being inflated, or by another method suitable for the balloon catheter 10, as shown in FIG. 6. In the high-profile operating mode, the lumen 12 of the balloon catheter has a diameter greater than the maximum diameter dₙ of the conical portion of the nosecone 200 and the optional collar 208. For instance, the lumen 12 may have a diameter of about 2 mm or 0.078 inches. The nosecone 200 may then be removed from the lumen 12 without disturbing the balloon catheter 10 when it is in the high-profile operating mode, such as by pulling the pullwire 206 discussed above.

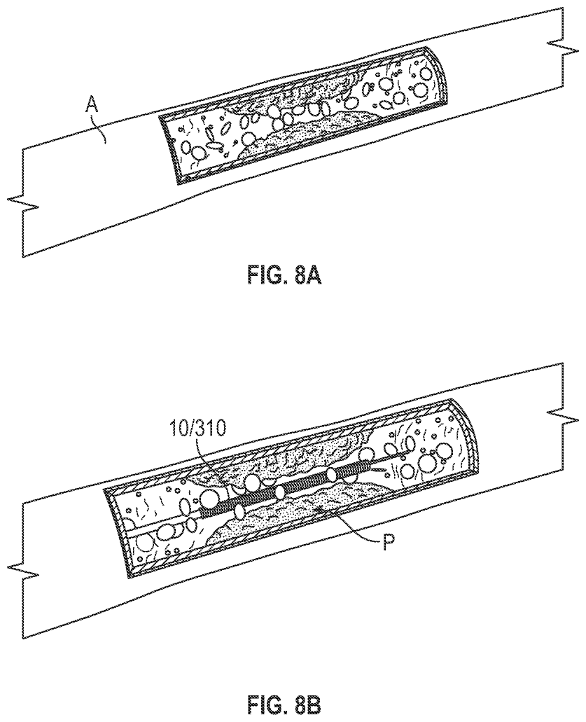

FIGS. 7A-D schematically show an example balloon catheter 310 which could be used with any balloon catheter system, including those systems 8/108 described above. The balloon catheter 310 in this example includes a balloon 310a wound into a helix 312. A cover 314 constrains the balloon 310a in the helix 312. The cover 314 also constrains the helix 312 from appreciably lengthening or shortening and inhibits acute bending or kinking of the balloon 310 while the balloon catheter is in use. The cover 314 can be provided on an inner surface 312a of the helix 312 (e.g., the surface facing the lumen 12), an outer surface 312b of the balloon catheter 10, or both. In the case where the cover 14 is provided on both surfaces 312a/312b, it encases the balloon 310 as best shown in FIG. 7D.

In some examples, the cover 314 may be fabricated by electrospinning. In general, electrospinning creates fibers, in particular small diameter fibers on the order of hundreds of nanometers in diameter. The fiber can be used to create a material sheet such as the cover 314. Electrospinning involves an electrohydrodynamic process, during which a liquid droplet is electrified to generate a jet, followed by stretching and elongation to generate fiber(s).

In some examples, the cover 314 may include any material suitable for electrospinning, including medical grade polymers such as polyethylene, polypropylene, polyurethane, nylon, nylon blends, and polyesters (such as polyethylene terephthalate (PET or PETE)).

The cover 314 can be compliant, semi-compliant, or rigid. A cover 314 that is compliant or semi-compliant can stretch with the balloon 310a as it is inflated to higher pressures.

The cover 314 may have hydrophilic or hydrophobic properties. A balloon catheter 310 with a hydrophilic cover 314 may be particularly beneficial for inserting a balloon catheter 310, or enabling the balloon catheter 310 to interface with other medical devices such as a (coronary) guidewire or guide catheter. In some examples, medical devices with a hydrophilic surface may be more slippery and easier to insert into a patient.

The cover 314 may be made from a material that has an additive, such as a pharmaceutical or drug-additive. For instance, the drug additive may be selected to prevent or reduce neointimal hyperplasia, which is also known as scar tissue. The balloon catheter 310 with cover 314 may therefore prevent or reduce scar tissue formation and keep a vessel or artery open for (blood) flow. Other drug additives are also contemplated. An additive may be used to treat a tumor or cancer, for example.

In another example, an additive may be selected to improve the properties of the balloon catheter 310/cover 314. For instance, the cover may be made with different additives. The cover 314 may include an additive that enables adhesion or bonding to the balloon 310a. In a particular example the additive may be an acrylic that allows for bonding. The additives disclosed herein may also be utilized with other balloon catheters, including the balloon catheter 10 examples disclosed or any other balloon catheters.

In some examples the balloon catheters 10/310 disclosed herein, or any other balloon catheters, may be made of materials such as PET, Pebax, Nylon, polyurethanes or a combination thereof or of any other compliant, non-compliant or semi-compliant material. In some examples, the balloon catheters 10/310, or any other balloon catheters, may have one or more medical or therapeutic products such as drugs, biologics or a combination thereof combined with a drug delivery matrix coated on its outermost surface or dispersed throughout or on parts of its body, which is made up of a combination of hydrophilic or hydrophobic or amphipathic excipients. The drug delivery matrix may include immediate release and delayed or controlled release matrices to achieve an efficacious and targeted delivery profile of the therapeutic product in the deployed environment.

In some examples, the balloon catheters 10/310 disclosed herein, or any other balloon catheters, may be deployed such that their residence time within the deployment environment can be as long as a few seconds, minutes, hours or longer so as to ensure delivery of the therapeutic product in the deployment environment at an optimal rate such that the efficacy level of the drug in the body can be reached at the lowest drug loading possible. In one embodiment of such a device, drugs like sirolimus are delivered at a surface loading ranging from near zero to less than 1 micrograms per cubic millimeter by manipulating the residence time, and drugs like Paclitaxel are delivered at a surface loading less than 2 micrograms per cu. mm to reduce the risks of particle embolization. For balloon catheters that provide a lumen 12 so as to not occlude blood flow when in a high profile operating mode, deployment time may be longer than in other types of balloon catheters in some examples. A balloon catheter that dos not provide such occlusion may be referred to as a perfusion balloon catheter.

The cover 314 may have a thickness that is suitable to constrain the helix as discussed above. In some examples, the cover 314 may have a thickness of about 10-about 100 microns (0.010-0.100 millimeters). In some examples, the cover 314 can be about 15 about 30 microns thick.

The cover 314 may have a porosity suitable for the application(s) of the balloon catheter. The porosity may be controlled during application/deposition of the cover 314 onto the balloon catheter 310. A cover 314 with appreciable porosity may act as a filter whereas a cover 314 with relatively lower porosity would not allow fluid flow therethrough. A lower porosity cover 314 may therefore be suitable for a procedure that aims to stop bleeding from an artery because the cover 314 does not allow blood flow through itself, such as treating a dissection or perforation of an artery or for closing a percutaneous access site into an artery. For instance, TAVR (transcatheter aortic valve replacement) procedures make large bore (diameter) access sites in an artery. When the TAVR procedure is done, the access site must be closed. If the access site is not closed, it will bleed and lead to adverse medical complications. A balloon catheter 310 with low porosity cover 314 can be inflated inside the artery to cover the access site and stop bleeding from the access site. The balloon catheter 310 may be kept in place until the artery naturally closes the access site and achieves hemostasis. In some instances, this may take about 30-60 minutes.

The cover 314 may include one or more sub-parts. For example, the cover 314 may include a base portion 314a associated with the inner surface 312a of the helix and an outer portion 314b associated with the outer surface 312b of the helix 312, shown in FIG. 7D. In this example, the portions 314a/314b of the cover 314 can be applied to the balloon catheter 310 separately and in sequence. For instance, the base portion 314a can be electrospun onto the inner surface 312a of the helix. The outer portion 314b can then be electrospun onto the outer surface 312b of the helix. Though the sub-parts 314a/314b may be applied separately, once applied the sub-parts 314a/314b can form a continuous or unitary cover 314 in some examples.

The helix 312 may form a number of adjacent coils C. In some examples, as shown in FIG. 7D, adjacent coils C provide an axial space S therebetween, and the cover 314 extends across the spaces S. The cover may include bridge portions 314c that extend axially across the space S. The bridge portions may be formed when applying the base portion 314a, the outer portion 314b, or both.

In some examples, the cover 314 may be fabricated by different methods. For instance, the cover 314 may be made using a dip coating method. In this example, the tubular balloon 310a that is formed in the helix 312 can be dipped into a source of liquid polymer. The polymer would dry to a solid state forming a cover 314 for the balloon catheter 310. A urethane-based polymer or other elastomers may be particularly suitable for a dip coated cover 14. Alternately, the cover 314 may be created using a vapor deposition method. This method creates a conformal coating which would constitute the cover 314 over the balloon 310a. A parylene-based polymer may be particularly suitable for vapor deposition of the cover 314.

The systems disclosed herein may be utilized with any of the methods and procedures disclosed in U.S. Pat. No. 11,096,813, the entire disclosure of which is hereby incorporated by reference.

A method of performing angioplasty may be performed with any of the systems disclosed herein. Fewer or additional steps than are recited herein could be performed within the scope of this disclosure, and the recited order of steps is not intended to limit this disclosure. The method may include accessing an artery. The artery may be accessed transradially or transfemorally in some examples. The method may include inserting a balloon 10/310 into to the artery in a low-profile operating mode. The balloon 10/310 may be constrained in a generally helical shape. The method may include situating the balloon 10/310 adjacent to a blockage in the artery. In some examples, the balloon 10/310 may be introduced at or near an area in an artery having plaque. The method may include forming a lumen 12 within the generally helical shape by expanding the balloon into a high-profile operating mode in which an inner surface of the helical shape defines the lumen 12. In some examples, the method may include passing a stent directly through the lumen 12 such that there is no intervening structure between the inner surface of the helical shape and the stent. The method may include implanting the stent into the blockage. In some examples, the balloon 10/310 may be removed before the stent is introduced.

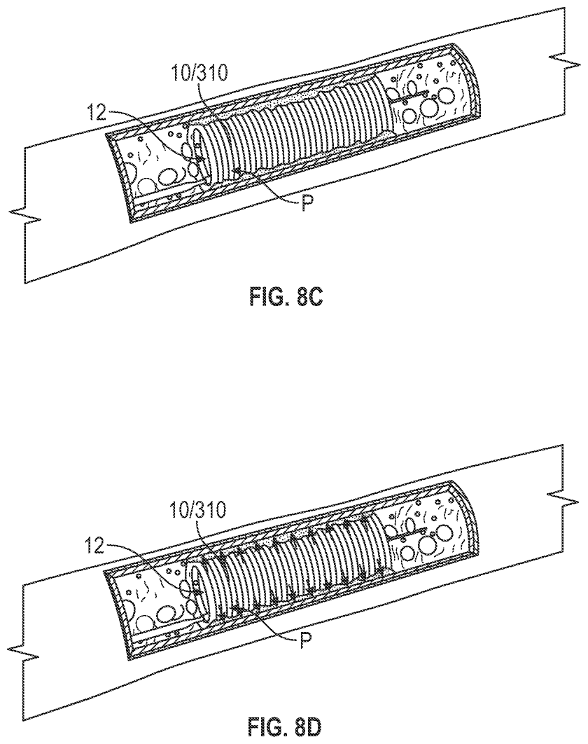

As shown in FIG. 8A, an example method may include an artery A having a plaque location P. As shown in FIG. 8B, a balloon catheter 10/310 may be introduced to the location P in a low-profile mode. In some examples, the balloon catheter 10/310 is introduced to the location according to any of the introduction methods disclosed herein. As shown in FIG. 8C the balloon is then converted into its high-profile mode at the location P. During high-profile mode, blood is still able to flow through the lumen 12. As shown in FIG. 8D, one or more medical and/or therapeutic products may be released from the balloon catheter 10/310 at the location P, during which blood is still able to flow through the lumen 12. In some examples, a stent (not shown) may be placed at the location P. In some examples, the stent may be placed at location P after the balloon 10/310 is removed from the location P. In some examples, the balloon 10/310 may be placed at a location spaced from the location P, converted to high-profile mode, and a stent may be passed through the lumen 12 on its way to being introduced to the location P.

A medical device according to one or more examples of this disclosure may be said to include a balloon catheter having a balloon positioned on the balloon catheter. The balloon may include compliant, semi-compliant or non-complaint material that creates a lumen. In a deflated state, the balloon may be transported to a point of application. The balloon may have one or more medical and/or therapeutic products, such as drugs, biologics or a combination thereof in some examples, combined with a drug delivery matrix coated or located on the balloon or dispersed through the balloon material. The therapeutic product may be released at a desired rate in the deployed environment. In some examples, the deployment rate can be optimized by adjusting the residence time of the balloon in the deployed area to seconds, minutes, hours or longer.

In some examples, the inflatable balloon is a helical shape including compliant, semi-compliant or non-complaint material, that can be coiled around a central axis and inflated to form a helix and create a lumen therethrough, and it can be transported, such as in a deflated state, to a point of application. In some examples, the helical balloon may have one or more medical or therapeutic products such as drugs, biologics or a combination thereof combined with a drug delivery matrix coated or located on the outer surface of the balloon or dispersed through the helix material, such that the therapeutic product can be released at a desired rate in the deployed environment and such rate can be optimized by adjusting the residence time of the balloon in the deployed area to seconds, minutes, hours or longer.

A method according to one or more examples of this disclosure may be said to include passing a balloon catheter in a deflated state over a wire to a location, in some examples the location being at or near a perforation, lesion, or other target location in a blood vessel. The method may include inflating the balloon to an appropriate pressure to match the general shape or size of the blood vessel and achieving contact with vessel at the target location for a certain residence time. In some examples, during the residence time, an encapsulating matrix releases the drug at the target location, while allowing for blood flow. The method may further include, after treatment, deflating the balloon by withdrawing the fluid from the balloon and retracting the balloon and the catheter from the blood vessel.

As used herein, the term “about” has the typical meaning in the art, however in a particular example “about” can mean deviations of up to 10% of the values described herein.

Although the different examples are illustrated as having specific components, the examples of this disclosure are not limited to those particular combinations. It is possible to use some of the components or features from any of the embodiments in combination with features or components from any of the other embodiments.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method of using a balloon catheter system, comprising:
advancing a balloon catheter and a nosecone together through a vasculature of a patient to a target location while the balloon catheter is in a low-profile operating mode, wherein the nosecone is disposed within a lumen of the balloon catheter;
transitioning the balloon catheter from the low-profile operating mode to a high-profile operating mode in which the lumen expands to a diameter greater than a maximum diameter of the nosecone; and
removing the nosecone from the lumen and from the patient while the balloon catheter remains in the high-profile operating mode at the target location.

22. The method of claim 21, wherein the nosecone comprises a conical portion spaced distally from an introducer portion and having an atraumatic tip.

23. The method of claim 22, wherein the introducer portion comprises a rod extending proximally from the conical portion.

24. The method of claim 23, wherein the removing step includes pulling a pullwire extending from the rod while the balloon catheter remains at the target location.

25. The method of claim 22, wherein the balloon catheter is collapsed around the introducer portion during the advancing step.

26. The method of claim 21, wherein the removing step includes pulling a pullwire extending from the nosecone while the balloon catheter remains at the target location.

27. The method of claim 21, wherein the balloon catheter comprises a therapeutic coating configured to deliver a therapeutic agent to tissue at the target location.

28. The method of claim 21, wherein the nosecone includes a conical portion having a maximum diameter approximately equal to or slightly larger than an outer diameter of the balloon catheter in the low-profile operating mode.

29. The method of claim 28, wherein a maximum diameter of the conical portion is about 0.040 inches to about 0.042 inches.

30. The method of claim 22, wherein the introducer portion includes a collar positioned at a proximal end thereof, wherein the collar has a diameter approximately equal to or slightly larger than an outer diameter of the balloon catheter in the low-profile operating mode.

31. The method of claim 22, wherein the introducer portion includes a collar positioned at a proximal end thereof, wherein the collar has a diameter substantially equal to a maximum diameter of the conical portion.

32. The method of claim 21, wherein the advancing the balloon catheter comprises advancing the balloon catheter over a guidewire extending through a lumen of the nosecone.

33. A balloon catheter system, comprising:
a balloon catheter having a lumen and being operable between a low-profile operating mode and a high-profile operating mode; and
a nosecone receivable within the lumen and including an introducer portion extending from a conical portion,
wherein, in the low-profile operating mode, the balloon catheter is configured to be disposed around the introducer portion, and
wherein, in the high-profile operating mode, the lumen has a diameter greater than a maximum diameter of the nosecone such that the nosecone is removable from the lumen and from a patient while the balloon catheter remains positioned within the patient.

34. The balloon catheter system of claim 33, wherein the conical portion includes an atraumatic distal tip.

35. The balloon catheter system of claim 33, wherein the introducer portion comprises a rod extending proximally from the conical portion, and wherein the balloon catheter is configured to be disposed around the rod in the low-profile operating mode.

36. The balloon catheter system of claim 33, wherein the nosecone further comprises a pullwire extending proximally from the nosecone and configured to permit removal of the nosecone from the lumen independently of the balloon catheter.

37. The balloon catheter system of claim 33, wherein the conical portion has a maximum diameter approximately equal to or slightly larger than an outer diameter of the balloon catheter in the low-profile operating mode.

38. The balloon catheter system of claim 37, wherein the maximum diameter of the conical portion is about 0.040 inches to about 0.042 inches.

39. The balloon catheter system of claim 33, wherein the introducer portion includes a collar positioned at a proximal end thereof, the collar having a diameter approximately equal to or slightly larger than an outer diameter of the balloon catheter in the low-profile operating mode such that the balloon catheter is axially trapped between the conical portion and the collar.

40. The balloon catheter system of claim 33, wherein the balloon catheter comprises a therapeutic coating configured to deliver a therapeutic agent to tissue when the balloon catheter is in the high-profile operating mode.

41. The balloon catheter system of claim 33, wherein the introducer portion includes at least one marker for visualization of the balloon catheter system using medical imaging.

42. The balloon catheter system of claim 41, wherein the at least one marker includes a first radio-opaque marker band and a second radio-opaque marker band.
